(19) United States
(12) Patent Application Publication
(10) Pub. No.: US 2023/0133942 A1
(43) Pub. Date: May 4, 2023

(54) KNEE JOINT ENDOPROSTHESIS

(71) Applicant: Aesculap AG, Tuttlingen (DE)

(72) Inventor: Berna Richter, Muehlheim (DE)

(21) Appl. No.: 18/090,542

(22) Filed: Dec. 29, 2022

Related U.S. Application Data
(63) Continuation of application No. PCT/EP2021/069466, filed on Jul. 13, 2021.

(30) Foreign Application Priority Data
Jul. 14, 2020 (DE) 10 2020 118 499.9

(51) Int. Cl. A61F 2/38

(52) U.S. Cl. CPC A61F 2/3868

(57) ABSTRACT

A knee joint endoprosthesis set for replacing a knee joint of a patient, a patient knee joint endoprosthesis set, and a knee joint endoprosthesis system. The knee joint endoprosthesis set includes a knee joint endoprosthesis with a femoral component, a tibial component, and a first meniscal component. The knee joint endoprosthesis set also includes a second meniscal component configured differently from the first meniscal component. One of the first and second meniscal components is arranged between the femoral component and the tibial component for forming the knee joint endoprosthesis.

### CROSS-REFERENCE TO RELATED APPLICATIONS

This application is a continuation of International Application No. PCT/EP2021/069466, filed on Jul. 13, 2021, and claims priority to German Application No. 10 2020 118 499.9, filed on Jul. 14, 2020. The contents of International Application No. PCT/EP2021/069466 and German Application No. 10 2020 118 499.9 are incorporated herein by reference in their entireties and for all purposes.

### FIELD

The present disclosure relates to knee joint endoprosthesis sets generally, and more specifically to a knee joint endoprosthesis set for replacing a knee joint of a patient, comprising a knee joint endoprosthesis with a femoral component, a tibial component, and a meniscal component.

Furthermore, the present disclosure relates patient knee joint endoprosthesis sets to generally, and more specifically to a patient knee joint endoprosthesis set for replacing any knee joint of the two knee joints of a patient.

Moreover, the present disclosure relates to knee joint endoprosthesis systems generally, and more specifically to a knee joint endoprosthesis system, comprising at least one knee joint endoprosthesis set for replacing a knee joint of a patient.

### BACKGROUND

Knee joint endoprostheses are typically implanted in patients that have a natural knee joint which is so severely damaged by wear or a trauma that the patient is significantly limited in their mobility and often is able to significantly load the knee joint only after taking strong pain medications.

As described at the outset, a knee joint endoprosthesis typically comprises a femoral component, which after resecting the natural knee joint is fixed to the correspondingly prepared femur, a tibial component, which is fixed to the correspondingly prepared tibia, and a meniscal component arranged between the femoral component and the tibial component. This serves in cooperation with the femoral component for forming a sliding pairing. When bending the knee joint endoprosthesis, which forms an artificial knee joint, the femoral component and the meniscal component slide on one another with contacting faces.

Furthermore, it is known that in a portion of patients the femur moves medially on the tibia significantly less than laterally upon a movement of the knee. An axis of rotation of the femur in the longitudinal direction of the leg thereby lies more on the medial side of the tibia. This is also referred to as “medial pivot”. In order to replace damaged natural knee joints with this movement pattern, knee joint endoprostheses are used that correspond to this “medial pivot” design.

Furthermore, studies shows that there are also patients with a movement pattern in which the femur moves laterally on the tibia significantly less than medially upon a movement of the knee. This is also referred to as “lateral pivot”. Correspondingly, the axis of rotation of the femur in the direction of the leg longitudinal axis thus lies more on the lateral side of the tibia. For treating this movement pattern, there are corresponding knee joint endoprostheses that correspond to this “lateral pivot” design.

So that after resecting the damaged knee a surgeon can still freely determine which kind of knee joint endoprosthesis to use, i.e., for example, a knee joint endoprosthesis with a design either in the form “medial pivot” or “lateral pivot”, two complete knee joint endoprostheses must thus be provided, including two fully equipped instrument sets. These thus comprise together at least six components, namely two femoral components, two meniscal components, and two tibial components of the required sizes. These, including all instruments and test implants, must all be provided for the surgical procedure. If they are not used and have been left in their sterile packaging, they can be used in a further surgical procedure. However, the test implants and instruments corresponding to the components that were not used must be elaborately reprocessed before being reused.

### SUMMARY

In a first aspect of the disclosure, a knee joint endoprosthesis set for replacing a knee joint of a patient, comprises a knee joint endoprosthesis with a femoral component, a tibial component, and a meniscal component. The knee joint endoprosthesis set comprises a further meniscal component, wherein the two meniscal components are configured differently. For forming the knee joint endoprosthesis, one of the two meniscal components is arranged between the femoral component and the tibial component cooperating therewith. The two meniscal components are configured mirror symmetrically to one another relative to a first mirror plane extending in a prosthesis longitudinal direction. The knee joint endoprosthesis set comprises a coupling device for coupling the tibial component and the two meniscal components in a coupling position. The coupling device is of mirror symmetrical configuration relative to a coupling device mirror plane extending in a prosthesis longitudinal direction. The femoral component comprises a condyle portion with a medial condyle and a lateral condyle. Each of the two meniscal components comprises a meniscal component sliding face cooperating with the two condyles with a medial sliding face region and a lateral sliding face region. The medial condyle and the medial sliding face region form a medial sliding pairing. The lateral condyle and the lateral sliding face region form a lateral sliding pairing. At least one of a shape and a size of the medial sliding face region and of the lateral sliding face region differ from one another. The condyle portion of the femoral component is of mirror symmetrical configuration relative to a femoral component mirror plane extending in a prosthesis longitudinal direction.

In a second aspect of the disclosure, a patient knee joint endoprosthesis set for replacing any knee joint of the two knee joints of a patient comprises:

a knee joint endoprosthesis set for replacing a knee joint of a patient. The knee joint endoprosthesis set comprises a knee joint endoprosthesis with a femoral component, a tibial component, and a meniscal component. The knee joint endoprosthesis set comprises a further meniscal component. The two meniscal components are configured differently. For forming the knee joint endoprosthesis, one of the two meniscal components is arranged between the femoral component and the tibial component cooperating therewith. The two meniscal components are configured mirror symmetrically to one another relative to a first mirror plane extending in a prosthesis longitudinal direction. The knee joint endoprosthesis set comprises a coupling device for coupling the tibial component and the two meniscal components in a coupling position. The coupling device is of mirror symmetrical configuration relative to a coupling device mirror plane extending in a prosthesis longitudinal direction. The femoral component comprises a condyle portion with a medial condyle and a lateral condyle. Each of the two meniscal components comprises a meniscal component sliding face cooperating with the two condyles with a medial sliding face region and a lateral sliding face region. The medial condyle and the medial sliding face region form a medial sliding pairing. The lateral condyle and the lateral sliding face region form a lateral sliding pairing. At least one of a shape and a size of the medial sliding face region and of the lateral sliding face region differ from one another. The condyle portion of the femoral component is of mirror symmetrical configuration relative to a femoral component mirror plane extending in a prosthesis longitudinal direction. The patient knee joint endoprosthesis set further comprises a further femoral component. The one of the two femoral components is configured in the form of a left femoral component and the other of the two femoral components is configured in the form of a right femoral component.

In a third aspect of the disclosure, a knee joint endoprosthesis system comprises:
a) at least one knee joint endoprosthesis set for replacing a knee joint of a patient. The at least one knee joint endoprosthesis set comprises a knee joint endoprosthesis with a femoral component, a tibial component, and a meniscal component. The at least one knee joint endoprosthesis set comprises a further meniscal component. The two meniscal components are configured differently. For forming the knee joint endoprosthesis, one of the two meniscal components is arranged between the femoral component and the tibial component cooperating therewith. The two meniscal components are configured mirror symmetrically to one another relative to a first mirror plane extending in a prosthesis longitudinal direction. The at least one knee joint endoprosthesis set comprises a coupling device for coupling the tibial component and the two meniscal components in a coupling position. The coupling device is of mirror symmetrical configuration relative to a coupling device mirror plane extending in a prosthesis longitudinal direction. The femoral component comprises a condyle portion with a medial condyle and a lateral condyle. Each of the two meniscal components comprises a meniscal component sliding face cooperating with the two condyles with a medial sliding face region and a lateral sliding face region. The medial condyle and the medial sliding face region form a medial sliding pairing. The lateral condyle and the lateral sliding face region form a lateral sliding pairing. At least one of a shape and a size of the medial sliding face region and of the lateral sliding face region differ from one another. The condyle portion of the femoral component is of mirror symmetrical configuration relative to a femoral component mirror plane extending in a prosthesis longitudinal direction.
   or
b) a patient knee joint endoprosthesis set for replacing any knee joint of the two knee joints of a patient. The patient knee joint endoprosthesis set comprises: a knee joint endoprosthesis set for replacing a knee joint of a patient. The knee joint endoprosthesis set comprises a knee joint endoprosthesis with a femoral component, a tibial component, and a meniscal component. The knee joint endoprosthesis set comprises a further meniscal component. The two meniscal components are configured differently. For forming the knee joint endoprosthesis, one of the two meniscal components is arranged between the femoral component and the tibial component cooperating therewith. The two meniscal components are configured mirror symmetrically to one another relative to a first mirror plane extending in a prosthesis longitudinal direction. The knee joint endoprosthesis set comprises a coupling device for coupling the tibial component and the two meniscal components in a coupling position. The coupling device is of mirror symmetrical configuration relative to a coupling device mirror plane extending in a prosthesis longitudinal direction. The femoral component comprises a condyle portion with a medial condyle and a lateral condyle. Each of the two meniscal components comprises a meniscal component sliding face cooperating with the two condyles with a medial sliding face region and a lateral sliding face region. The medial condyle and the medial sliding face region form a medial sliding pairing. The lateral condyle and the lateral sliding face region form a lateral sliding pairing. At least one of a shape and a size of the medial sliding face region and of the lateral sliding face region differ from one another. The condyle portion of the femoral component is of mirror symmetrical configuration relative to a femoral component mirror plane extending in a prosthesis longitudinal direction. The patient knee joint endoprosthesis set further comprises a further femoral component. The one of the two femoral components is configured in the form of a left femoral component and the other of the two femoral components is configured in the form of a right femoral component.

### BRIEF DESCRIPTION OF THE DRAWING FIGURES

The foregoing summary and the following description may be better understood in conjunction with the drawing figures, of which:

FIG. 1 shows an exploded depiction of a first embodiment of a knee joint endoprosthesis set;

FIG. 2 shows an exploded depiction of a further embodiment of a knee joint endoprosthesis set;

FIG. 3 shows a schematic depiction of the knee joint endoprosthesis set from FIG. 1 with a combination of two different meniscal components;

FIG. 4 shows a perspective view of the combination depicted on the left in FIG. 3;

FIG. 5 shows a view of a femoral component from the front;

FIG. 6 shows a side view of the femoral component from FIG. 5;

FIG. 7 shows a further view of the femoral component from FIG. 5;

FIG. 8 shows a view similar to FIG. 7 of a further embodiment of a femoral component;

FIG. 9 shows a side view of the tibial component from FIGS. 1 to 4;

FIG. 10 shows a plan view of the tibial component from FIG. 9;

FIG. 11 shows a side view of the meniscal component depicted on the left in FIG. 1;

FIG. 12 shows a plan view of the meniscal component from FIG. 11;

FIG. 13 shows a section view along line 13-13 in FIG. 11;

FIG. 14 shows a section view along line 14-14 in FIG. 11;

FIG. 15 shows a section view along line 15-15 in FIG. 11;

FIG. 16 shows a side view of the meniscal component depicted on the right in FIG. 1; and

FIG. 17 shows a plan view of the meniscal component from FIG. 16.

### DETAILED DESCRIPTION

Although the disclosure is illustrated and described herein with reference to specific embodiments, the disclosure is not intended to be limited to the details shown. Rather, various modifications may be made in the details within the scope and range of equivalents of the claims and without departing from the disclosure.

The present disclosure relates to a knee joint endoprosthesis set for replacing a knee joint of a patient, comprising:

a knee joint endoprosthesis with a femoral component, a tibial component, and a meniscal component,

wherein the knee joint endoprosthesis set comprises a further meniscal component, wherein the two meniscal components are configured differently, wherein for forming the knee joint endoprosthesis, one of the two meniscal components is arranged between the femoral component and the tibial component cooperating therewith,

wherein the two meniscal components are configured mirror symmetrically to one another relative to a first mirror plane extending in a prosthesis longitudinal direction,

wherein the knee joint endoprosthesis set comprises a coupling device for coupling the tibial component and the two meniscal components in a coupling position, wherein the coupling device is of mirror symmetrical configuration relative to a coupling device mirror plane extending in a prosthesis longitudinal direction,

wherein the femoral component comprises a condyle portion with a medial condyle and a lateral condyle, wherein each of the two meniscal components comprises a meniscal component sliding face cooperating with the two condyles with a medial sliding face region and a lateral sliding face region, wherein the medial condyle and the medial sliding face region form a medial sliding pairing, wherein the lateral condyle and the lateral sliding face region form a lateral sliding pairing,

wherein at least one of a shape and a size of the medial sliding face region and of the lateral sliding face region differ from one another, and wherein the condyle portion of the femoral component is of mirror symmetrical configuration relative to a femoral component mirror plane extending in a prosthesis longitudinal direction.

Further developing a knee joint endoprosthesis set of the kind described at the outset in the proposed manner has the advantage, in particular, that two complete knee joint endoprostheses, including all associated instruments and test implants, as the case may be, do not have to be provided in order to achieve different movement patterns, but instead only four components, namely a femoral component, a tibial component, and two meniscal components. Different movement patterns of the artificial knee joint to be implanted, i.e., of the knee joint endoprosthesis, can be achieved through the selective use of the one or the other meniscal component. Selectively means that only one, i.e., only a single one, of the two meniscal components is positioned between the tibial component and the femoral component for forming the knee joint endoprosthesis. This has the advantage, as mentioned, that only four components have to be provided for a procedure to enable a surgeon full freedom and flexibility during the surgical procedure, in particular even after the bone has been sawed or brought into shape. This is not possible with knee prosthesis systems that are currently available, because until now when a surgeon had decided on a knee variant and had prepared the bone accordingly, there was no longer an option to switch to a different knee prosthesis system without additionally removing healthy bone. Due to the smaller number of required components of the knee joint endoprosthesis set further developed as proposed, expenditure in the production and in the reprocessing of unused components of the knee joint endoprosthesis set can be significantly reduced. In the knee joint endoprosthesis set further developed as proposed, the number of components that are absolutely necessary can thus be reduced by two, which corresponds to a reduction by a third compared to two knee joint endoprostheses to be made completely available, which each can be used exclusively for one single movement pattern, for example a movement pattern as described at the outset that corresponds to a “medial pivot” and to a “lateral pivot” on the other hand. For achieving these movement patterns, it is advantageous, in particular, if the two meniscal components, i.e., independently of which is selected for forming the knee joint endoprosthesis and is arranged between the tibial component and the femoral component, are immovably fixable to the tibial component. Such a knee joint endoprosthesis thus has a so-called “fixed bearing”, a meniscal component that is immovably held on the tibial component after implantation. Said knee joint endoprosthesis is thus of the “fixed bearing” type. With such a prosthesis type, due to the immovable mounting of the selected meniscal component on the tibial component, only a direct or immediate relative movement between the femoral component and the selected meniscal component is made possible.

It is favorable if the knee joint endoprosthesis set comprises only one single femoral component and one single tibial component. The number of components comprised by the knee joint endoprosthesis set can thus be limited to a total number of four components, namely the single femoral component, the single tibial component, and two different meniscal components. The two different meniscal components make it possible, as already explained, to achieve with this knee joint endoprosthesis set comprising four components, for example, two different movement patterns for a knee joint endoprosthesis, in particular if it is of the “fixed bearing” type. This is achieved by appropriately selecting the meniscal component.

It is advantageous if the knee joint endoprosthesis set comprises only two meniscal components. Thus, as mentioned, the number of components of the knee joint endoprosthesis set in total can be limited to four components. Nonetheless, it is possible with two different meniscal components, in particular, to achieve two different movement patterns for the knee joint endoprosthesis. It is possible, in particular, even after cutting the bone on the femur and/or tibia and after assessing the joint situation with appropriate test components, to still select or alter the kinematics, namely without additional effort or reworking on the bone. This is possible simply by swapping the meniscal components. In particular, different meniscal components can even be compared with one another intraoperatively in connection with test components.

In order to further simplify the setup of the knee joint endoprosthesis set, it may be advantageous if the two meniscal components are configured mirror symmetrically to one another relative to a first mirror plane extending in a prosthesis longitudinal direction. In other words, the one meniscal component can be converted into the other meniscal component by reflection on the first mirror plane. This is possible, in particular, even when the two meniscal components are not mirror symmetrical each seen on its own. The proposed configuration of the two meniscal components helps, in particular, to minimize design efforts, since a shape of the one meniscal component is transferable to the other meniscal component by a described reflection on the first mirror plane.

It is favorable if the knee joint endoprosthesis set comprises a coupling device for coupling the tibial component and the two meniscal components in a coupling position and if the coupling device is of mirror symmetrical configuration relative to a coupling device mirror plane extending in a prosthesis longitudinal direction. A design of that kind has the advantage, in particular, that the mirror symmetrically configured coupling device enables a selective coupling of both meniscal components to the same tibial component. It is thus sufficient in principle to provide one single tibial component, which then can be selectively coupled in a defined manner to each of the two meniscal components of the knee joint endoprosthesis set in order to form a complete knee joint endoprosthesis. In addition, the mirror symmetrically configured coupling device has the advantage that it requires less design effort. A prosthesis longitudinal direction may be defined, in particular, by a leg longitudinal axis when the knee joint endoprosthesis adopts an extended position, i.e., when the lower leg of the patient with the tibial component arranged thereon and the upper leg of the patient with the femoral component arranged thereon adopt an extended position, which is the case, e.g., when the patient stands or lies with the knee joint fully extended, i.e. with the leg as a whole extended. The coupling device mirror plane may furthermore extend in parallel to a median plane of the patient. Said plane may thus be spanned, e.g., by the leg longitudinal axis on the one hand and an axis extending in the anterior-posterior direction.

It is advantageous if the tibial component and one of the two meniscal components are coupled to one another in the coupling position to form the knee joint endoprosthesis in such a way that the one of the two meniscal components is immovably held on the tibial component. This further development makes it possible, in particular, to obtain a knee joint endoprosthesis of the so-called “fixed-bearing” type, i.e., a knee joint endoprosthesis in which the meniscal component is immovably held on the tibial component after implantation, in particular in a plane extending transversely to the prosthesis longitudinal direction.

It is favorable if the coupling device comprises two first coupling portions and a second coupling portion, if one of the two first coupling portions is arranged or formed on each of the two meniscal components, if the second coupling portion is arranged or formed on the tibial component, and if in the coupling position the first coupling portion of one of the two meniscal components and the second coupling portion are in engagement with one another in a force-locking and/or positive-locking manner and in a separating position are out of engagement. With such a coupling device, a defined connection between the tibial component and selectively one of the two meniscal components can be achieved in a simple manner. In particular, a surgeon can still change the desired movement pattern of the knee joint endoprosthesis during a surgical procedure by swapping the meniscal component.

The knee joint endoprosthesis set can be made of simple construction if the first coupling portion is of mirror symmetrical configuration relative to the coupling device mirror plane.

The second coupling portion is preferably of mirror symmetrical configuration relative to the coupling device mirror plane. This can help, for one, to minimize design effort of the knee joint endoprosthesis set. For another, a coupling to a corresponding first coupling portion that is also configured mirror symmetrically to the coupling device mirror plane can thus be optimized.

In accordance with a further preferred embodiment, provision may be made that the femoral component comprises a condyle portion with a medial condyle and a lateral condyle, that each of the two meniscal components comprises a meniscal component sliding face cooperating with the two condyles with a medial sliding face region and a lateral sliding face region, and that the medial condyle and the medial sliding face region form a medial sliding pairing, and that the lateral condyle and the lateral sliding face region form a lateral sliding pairing. A knee joint endoprosthesis set formed as described enables the formation of knee joint endoprostheses in which the femoral component can cooperate with each of the two meniscal components in a desired and defined manner. In particular, due to the different configuration of the meniscal components, for example the meniscal component sliding faces thereof, different movement patterns of the knee joint endoprosthesis can be achieved when the femoral component cooperates with the one or with the other meniscal component.

A shape and/or a size of the medial sliding face region and of the lateral sliding face region favorably differ from one another. Shape and size of the sliding face regions of the meniscal component sliding face make it possible, in particular, to achieve different movement patterns with the knee joint endoprosthesis in a defined manner. This can be achieved, in particular, by predetermining a shape of the respective sliding face regions, which then in cooperation with the respective femoral condyle, in particular, enable exclusively a rolling movement or exclusively a sliding movement or a combination of a rolling movement and a sliding movement.

The condyle portion of the femoral component is favorably of mirror symmetrical configuration relative to a femoral component mirror plane extending in a prosthesis longitudinal direction. This design makes it possible, in particular, to configure the medial condyle and the lateral condyle of the femoral component to be mirror symmetrical. This has the advantage, in particular, that a movement pattern of the knee joint endoprosthesis that can be formed with the knee joint endoprosthesis set is able to be predetermined exclusively by the meniscal component sliding face. It is thus possible, in particular, to alter a movement pattern of the knee joint endoprosthesis when the one meniscal component is replaced by the other meniscal component.

A construction of the knee joint endoprosthesis set can be simplified, in particular, by the coupling device mirror plane defining the femoral component mirror plane. Alternatively, it is also possible that the coupling device mirror plane and the femoral component mirror plane extend in parallel to one another. In this case, for example, an offset of the two condyles of the femoral component relative to the tibial component can be achieved when the two stated mirror planes do not coincide in an implantation position of the knee joint endoprosthesis, but instead are offset in parallel to one another.

It is favorable if the medial condyle and the medial sliding face region define a medial joint region, if the lateral condyle and the lateral sliding face region define a lateral joint region, and if one of the two joint regions has a higher congruency between the condyle and the associated sliding face region than the other. As a result of the described design, it is possible, in particular, to achieve different movement patterns of the knee joint endoprosthesis. For example, a region of high congruency can predetermine a preferred rotation about a rotation center or substantially such a rotation, and a less congruent joint region can predetermine a sliding movement or an overlapping sliding movement/rolling movement of the femoral component and the meniscal component cooperating therewith. The higher the congruency in the respective joint region is, the better a guidance and positioning of the femoral component and the meniscal component relative to one another in this joint region is.

The joint region with the higher congruency favorably defines a ball-jointed or substantially ball jointed joint region. This has the advantage, in particular, that the femoral component can be rotated relative to the meniscal component about the ball jointed joint region, for example even in an extended position of the knee joint, thereby making a rotation of the femoral component substantially about a leg longitudinal axis of the patient possible. Depending on whether the ball-jointed joint region is on the medial side or on the lateral side of the knee joint endoprosthesis, for example depending on whether the one or the other meniscal component is used, in particular, a corresponding knee joint endoprosthesis with a “medial pivot” design or a “lateral pivot” design can thus be achieved. The appropriate selection is still possible for a surgeon during the surgical procedure.

In accordance with a further preferred embodiment, provision may be made that the meniscal component of the knee joint endoprosthesis comprises a first coupling portion for coupling, in particular for immovably coupling, to the tibial component and a meniscal component sliding face cooperating with the femoral component with a medial sliding face region and a lateral sliding face region, that the first coupling portion is of mirror symmetrical configuration relative to a coupling portion mirror plane, and that the sliding face region is of asymmetrical configuration relative to the coupling portion mirror plane. This configuration may also be provided, in particular, in a knee joint endoprosthesis set of the kind described at the outset. The proposed further development simplifies, in particular, the design of the knee joint endoprosthesis set, because the symmetrical configuration of the first coupling portion enables any combination with tibial components that have a correspondingly configured, i.e. mirror symmetrical second coupling portion. In contrast, the asymmetrical configuration of the two sliding face regions relative to one another makes it possible to achieve a corresponding movement pattern with the knee joint endoprosthesis, for example according to the “medial pivot” design or according to the “lateral pivot” design. For achieving these movement patterns, it is advantageous, in particular, if the two meniscal components, as described above in detail, i.e., independently of which is selected for forming the knee joint endoprosthesis and is arranged between the tibial component and the femoral component, are immovably fixable to the tibial component for achieving a knee joint endoprosthesis of the “fixed-bearing” type.

It is favorable if a shape and/or a size of the medial sliding face region and the lateral sliding face region differ from one another. By predetermining shape and/or size of the sliding face regions, in particular, special movement patterns upon a flexion of the knee joint endoprosthesis can be predetermined.

Further, the disclosure relates to a patient knee joint endoprosthesis set for replacing any knee joint of the two knee joints of a patient, comprising:

a knee joint endoprosthesis set for replacing a knee joint of a patient, comprising a knee joint endoprosthesis with a femoral component, a tibial component, and a meniscal component, wherein the knee joint endoprosthesis set comprises a further meniscal component, wherein the two meniscal components are configured differently, wherein for forming the knee joint endoprosthesis, one of the two meniscal components is arranged between the femoral component and the tibial component cooperating therewith,

wherein the two meniscal components are configured mirror symmetrically to one another relative to a first mirror plane extending in a prosthesis longitudinal direction,

wherein the knee joint endoprosthesis set comprises a coupling device for coupling the tibial component and the two meniscal components in a coupling position, wherein the coupling device is of mirror symmetrical configuration relative to a coupling device mirror plane extending in a prosthesis longitudinal direction,

wherein the femoral component comprises a condyle portion with a medial condyle and a lateral condyle, wherein each of the two meniscal components comprises a meniscal component sliding face cooperating with the two condyles with a medial sliding face region and a lateral sliding face region, wherein the medial condyle and the medial sliding face region form a medial sliding pairing, wherein the lateral condyle and the lateral sliding face region form a lateral sliding pairing,

wherein at least one of a shape and a size of the medial sliding face region and of the lateral sliding face region differ from one another, and wherein the condyle portion of the femoral component is of mirror symmetrical configuration relative to a femoral component mirror plane extending in a prosthesis longitudinal direction;

the patient knee joint endoprosthesis set further comprising:

a further femoral component, wherein the one of the two femoral components is configured in the form of a left femoral component and wherein the other of the two femoral components is configured in the form of a right femoral component.

The proposed configuration of a patient knee joint endoprosthesis set makes it possible, in particular, to compose a knee joint endoprosthesis with two femoral components for a left and a right knee, two meniscal components, and a tibial component, which knee joint endoprosthesis is able to be implanted in order to replace one of the two knee joints of a patient. Such a patient knee joint endoprosthesis set with a total of five components therefore still has one component fewer than has been the case until now for knee joint endoprostheses in which at least three components have to be provided for each knee joint, even when it has already been decided whether a “medial pivot” design or a “lateral pivot” design is to be implemented for the respective knee joint. If in the case of conventional knee joint endoprosthesis systems the decision as to whether a “medial pivot” design or a “lateral pivot” design is to be implemented is to be made not until during the procedure, a total of at least 12 components must be provided. Therefore, for a knee joint endoprosthesis of the “medial pivot” design, at least three components are required, namely both for a left knee joint and for a right knee joint. Likewise, for a knee joint endoprosthesis of the “lateral pivot” design, at least three components are required, namely both for a left knee joint and for a right knee joint. In accordance with the disclosure, as described above, only five components are needed for this requirement.

The two femoral components and the two meniscal components are advantageously configured to be combinable with one another as desired. Combinable with one another as desired in this sense means, in particular, that each femoral component is selectively combinable with the two meniscal components. Thus, a total of four combinations can be achieved with the patient knee joint endoprosthesis set. For example, both a knee joint endoprosthesis for replacing the left knee and the right knee, each with a “medial pivot” design or a “lateral pivot” design, can be achieved. In particular when a decision as to which of the two knee joints of the patient is to be stabilized “medially” or “laterally” should remain open until shortly before the surgical procedure, such a patient knee joint endoprosthesis set has the advantage of actually being able to leave this decision open until the last moment. In other words, the kinematics of the knee joint endoprosthesis can be adapted up until completion of the procedure. Whether the left knee or the right knee is to be operated on first makes no difference. Due to the symmetry of the system, only the components required for forming a left and a right knee joint endoprosthesis have to be provided to the surgeon. This is ensured if they have at least two femoral components, two tibial components, and meniscal components available to them. Because the meniscal components from the right knee joint and left knee joint are interchangeable, with such a system the surgeon is thus able to support a “lateral pivot” design or a “medial pivot” design both in the left and in the right knee, depending on the combination.

It is favorable if the two femoral components are configured mirror symmetrically to one another relative to a second mirror plane extending in a prosthesis longitudinal direction. This means, in particular, that the one femoral component can be converted into the other femoral component by reflection on the second mirror plane. Design efforts in forming the patient knee joint endoprosthesis set can thus be minimized.

The first mirror plane preferably defines the second mirror plane. Thus, in particular, the two femoral components and the two meniscal components can be configured mirror symmetrically in the described manner relative to the first mirror plane and the second mirror plane, so that the femoral component cooperating with the one meniscal component can be converted into the other femoral component and the other meniscal component cooperating therewith by reflection on one of the two mirror planes.

The patient knee joint endoprosthesis set advantageously comprises a further tibial component. Such a patient knee joint endoprosthesis set thus comprises, in particular, two tibial components, two femoral components, and two meniscal components. In particular, the two meniscal components can be selectively coupled to the tibial component as described above. Thus, a total of, for example, two artificial knee joints can be formed, but with the advantage that the concrete design, i.e., for example “medial pivot” or “lateral pivot”, can still be selected during the implantation, namely by correspondingly swapping the two meniscal components.

In order to minimize the production expenditure of the patient knee joint endoprosthesis set, it is favorable if the tibial component comprised by the knee joint endoprosthesis set and the further tibial component are of identical configuration. Such a tibial component can be selectively fixed to the left or to the right tibia of a patient. Thus, as described, in principle only three components have to be constructed for the patient knee joint endoprosthesis set, namely a femoral component, a meniscal component, and a tibial component. The femoral component and the meniscal component can be converted into a corresponding counterpart for the other knee by, as described above, for example, reflecting once on a mirror plane, such that a total of five different components are obtained.

Further, the disclosure relates to a knee joint endoprosthesis system, comprising:
a) at least one knee joint endoprosthesis set for replacing a knee joint of a patient, comprising a knee joint endoprosthesis with a femoral component, a tibial component, and a meniscal component, wherein the at least one knee joint endoprosthesis set comprises a further meniscal component, wherein the two meniscal components are configured differently, wherein for forming the knee joint endoprosthesis, one of the two meniscal components is arranged between the femoral component and the tibial component cooperating therewith,
   wherein the two meniscal components are configured mirror symmetrically to one another relative to a first mirror plane extending in a prosthesis longitudinal direction,
   wherein the at least one knee joint endoprosthesis set comprises a coupling device for coupling the tibial component and the two meniscal components in a coupling position, wherein the coupling device is of mirror symmetrical configuration relative to a coupling device mirror plane extending in a prosthesis longitudinal direction,
   wherein the femoral component comprises a condyle portion with a medial condyle and a lateral condyle, wherein each of the two meniscal components comprises a meniscal component sliding face cooperating with the two condyles with a medial sliding face region and a lateral sliding face region, wherein the medial condyle and the medial sliding face region form a medial sliding pairing, wherein the lateral condyle and the lateral sliding face region form a lateral sliding pairing,
   wherein at least one of a shape and a size of the medial sliding face region and of the lateral sliding face region differ from one another, and wherein the condyle portion of the femoral component is of mirror symmetrical configuration relative to a femoral component mirror plane extending in a prosthesis longitudinal direction;
or
b) a patient knee joint endoprosthesis set for replacing any knee joint of the two knee joints of a patient, comprising:
   a knee joint endoprosthesis set for replacing a knee joint of a patient, the a knee joint endoprosthesis set comprising a knee joint endoprosthesis with a femoral component, a tibial component, and a meniscal component, wherein the knee joint endoprosthesis set comprises a further meniscal component, wherein the two meniscal components are configured differently, wherein for forming the knee joint endoprosthesis, one of the two meniscal components is arranged between the femoral component and the tibial component cooperating therewith,
   wherein the two meniscal components are configured mirror symmetrically to one another relative to a first mirror plane extending in a prosthesis longitudinal direction,
   wherein the knee joint endoprosthesis set comprises a coupling device for coupling the tibial component and the two meniscal components in a coupling position, wherein the coupling device is of mirror symmetrical configuration relative to a coupling device mirror plane extending in a prosthesis longitudinal direction, wherein the femoral component comprises a condyle portion with a medial condyle and a lateral condyle, wherein each of the two meniscal components comprises a meniscal component sliding face cooperating with the two condyles with a medial sliding face region and a lateral sliding face region, wherein the medial condyle and the medial sliding face region form a medial sliding pairing, wherein the lateral condyle and the lateral sliding face region form a lateral sliding pairing,
   wherein at least one of a shape and a size of the medial sliding face region and of the lateral sliding face region differ from one another, and wherein the condyle portion of the femoral component is of mirror symmetrical configuration relative to a femoral component mirror plane extending in a prosthesis longitudinal direction, and
   wherein the patient knee joint endoprosthesis set further comprises a further femoral component, wherein the one of the two femoral components is configured in the form of a left femoral component and wherein the other of the two femoral components is configured in the form of a right femoral component.

Such a knee joint endoprosthesis system may, in particular, also comprise two, three, or more knee joint endoprosthesis sets or patient knee joint endoprosthesis sets. For example, they may be made of different materials. In particular, for preparing an implantation of a knee joint endoprosthesis, a surgeon can select from such a knee joint endoprosthesis system the components that are optimally suited for the patient, in particular with respect to size.

It is advantageous if the knee joint endoprosthesis system comprises at least two knee joint endoprosthesis sets and if the femoral component and/or the two meniscal components and/or the tibial component of the at least two knee joint endoprosthesis sets differ in shape and/or size. With such a knee joint endoprosthesis system, in particular, knee joint endoprostheses of different shapes and movement designs can be achieved in order to, in particular, equip patients of different sizes with suitable prostheses. For example, such a knee joint endoprosthesis system makes it possible to implant a knee joint endoprosthesis, the movement pattern of which is adapted to the natural movement pattern of the replaced knee joint of the patient.

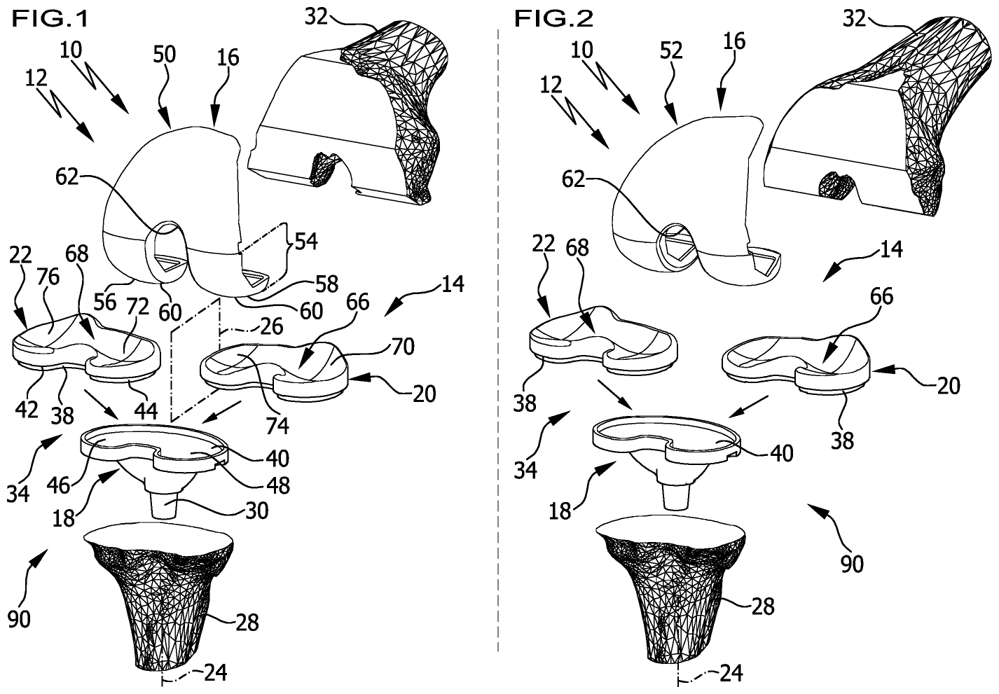

An embodiment of a knee joint endoprosthesis set 10 is schematically depicted in FIG. 1. It forms part of a knee joint endoprosthesis system 12.

The knee joint endoprosthesis set 10 serves to replace a damaged knee joint of a patient. It comprises a knee joint endoprosthesis 14 with a femoral component 16, a tibial component 18, and two meniscal components 20 and 22.

The two meniscal components 20 and 22 are configured differently. For forming the knee joint endoprosthesis 14, one of the two meniscal components 20, 22 is arranged between the femoral component 16 and the tibial component 18 and cooperates therewith.

The knee joint endoprosthesis set 10 comprises only one single femoral component 16 and one single tibial component 18. Furthermore, it comprises exclusively the two meniscal components 20 and 22.

The two meniscal components 20 and 22 are configured mirror symmetrically to one another relative to a first mirror plane 26 extending in a prosthesis longitudinal direction 24. The prosthesis longitudinal direction is defined by a longitudinal axis of the leg, which, in particular, is defined by a longitudinal axis of the femur 28 of the patient on which the tibial component 18 is arranged. A shank 30 of the tibial component 18 extends in parallel or substantially in parallel to the prosthesis longitudinal direction 24.

The described mirror symmetrical configuration of the two meniscal components 20 and 22 means, in particular, that the meniscal component 20 can be converted into the meniscal component 22 and vice versa by reflection on the first mirror plane 26.

The femoral component 16 is configured to be fixed to a prepared femur 32 of a patient.

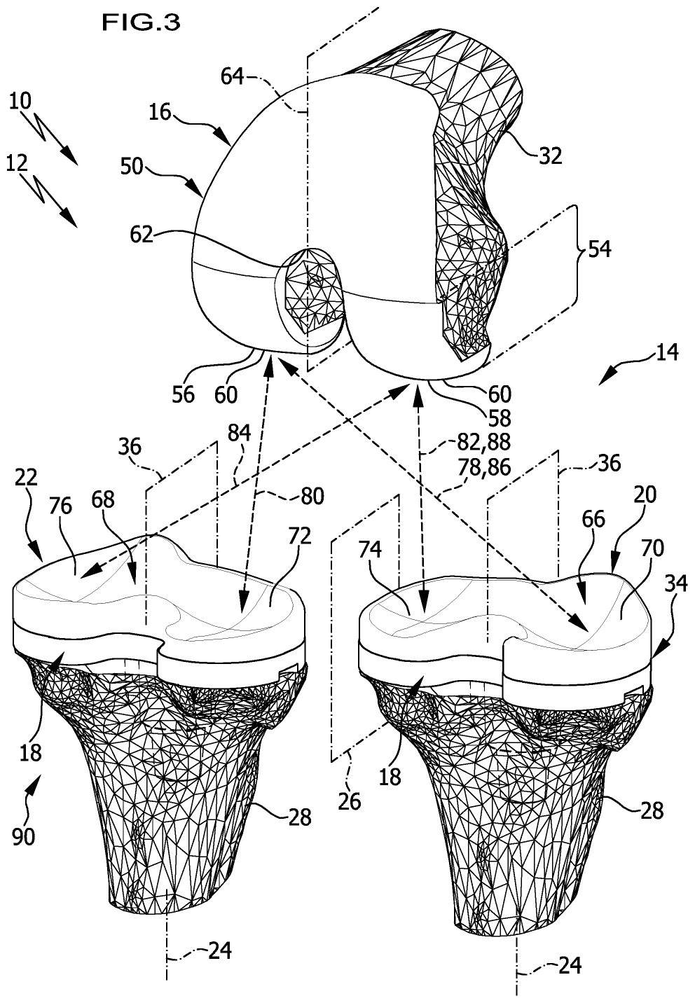

The knee joint endoprosthesis set 10 comprises a coupling device 34 for coupling the tibial component 18 and the meniscal component 20 or 22 in a coupling position. The coupling position is schematically depicted in FIG. 3.

In the coupling position, one of the two meniscal components 20 and 22 is immovably coupled to the tibial component. The knee joint endoprosthesis 14 thus forms an artificial knee joint of the “fixed bearing” type.

The coupling device 34 is of mirror symmetrical configuration relative to a coupling device mirror plane 36 extending in the prosthesis longitudinal direction 24.

The coupling device 34 comprises two first coupling portions 38 and a second coupling portion 40. The two first coupling portions 38 are each arranged or formed on one of the two meniscal components 20 and 22. They are configured in the form of flat kidney-shaped projections 44, which in the coupling position engage in a positive-locking manner into a corresponding recess 46 on the tibial component 18.

The recess 46 defines a planar base face 48, which extends transversely, namely perpendicularly, to the prosthesis longitudinal direction 24 and faces in the direction toward the meniscal component 20 or 22.

FIG. 1 shows schematically the knee joint endoprosthesis set 10 in which the coupling portions 38 and 40 are out of engagement. Depicted schematically in FIG. 3 in the lower region are coupling positions of the two meniscal components 20 and 22, each with one of two identically configured tibial components 18.

As already explained, the coupling device 34 is of mirror symmetrical configuration relative to the coupling device mirror plane 36. Likewise, the coupling portions 38 and 40 are of mirror symmetrical configuration relative to the coupling device mirror plane 36. This makes it possible to couple the tibial component both to the meniscal component 20 and to the meniscal component 22. Which of the two meniscal components 20 and 22 is to be used to form the knee joint endoprosthesis 14 depends, in particular, on which movement pattern is to be reproduced with the knee joint endoprosthesis 14. This is explained in more detail in the following.

FIGS. 1 and 3 shows a femoral component 16 that is configured in the form of a left femoral component 50. A right femoral component 52 is depicted schematically, in particular, in FIGS. 2 and 8.

The femoral component 16 comprises a condyle portion 54 with a medial condyle 56 and a lateral condyle 58. The two condyles 56 and 58 each have a crowned outer face 60. The outer face 60 is spherically shaped at least in partial regions.

The condyles 56 and 58 are separated from one another by a notch 62.

The condyle portion 54 of the femoral component 16 is of mirror symmetrical configuration relative to a femoral component mirror plane 64 extending in the prosthesis longitudinal direction 24.

In the embodiments depicted in the Figures, the coupling device mirror plane 36 defines the femoral component mirror plane 64.

Each of the two meniscal components 20 and 22 defines a respective meniscal component sliding face 66 and 68 with a respective medial sliding face region 70 and 72 and a respective lateral sliding face region 74 and 76.

The knee joint endoprosthesis set 10 is configured in such a way that the medial condyle 56 together with one of the two medial sliding face regions 70 or 72 forms a medial sliding pairing 78 or 80. The lateral condyle 58 together with one of the lateral sliding face regions 74 or 76 forms a lateral sliding pairing 82 or 84. The association of the condyles 56 and 58 with the sliding face regions 70 and 72 is indicated schematically in FIG. 3 by the double arrows symbolizing the sliding pairings 78, 80 and 84, 86.

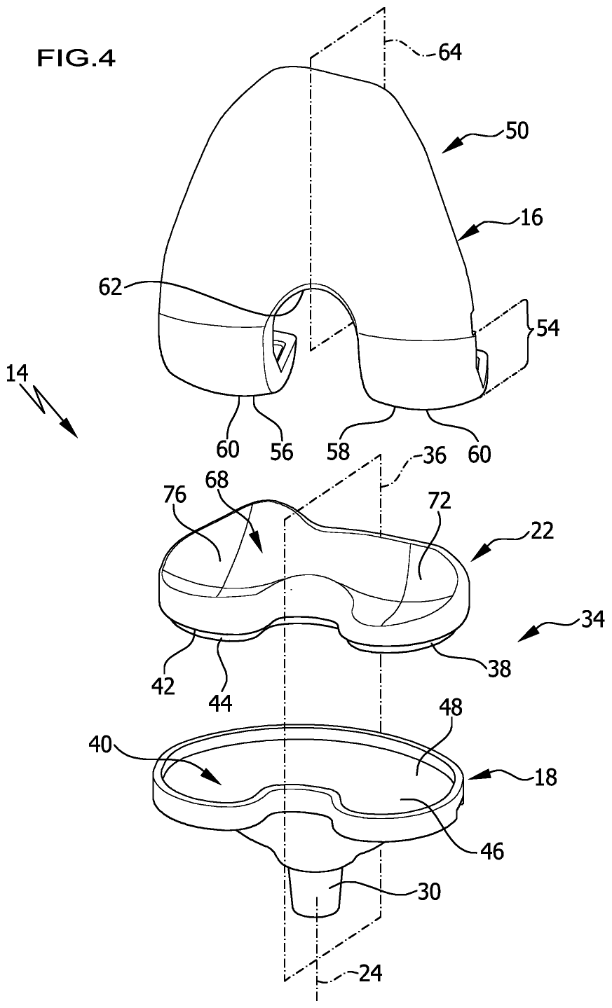

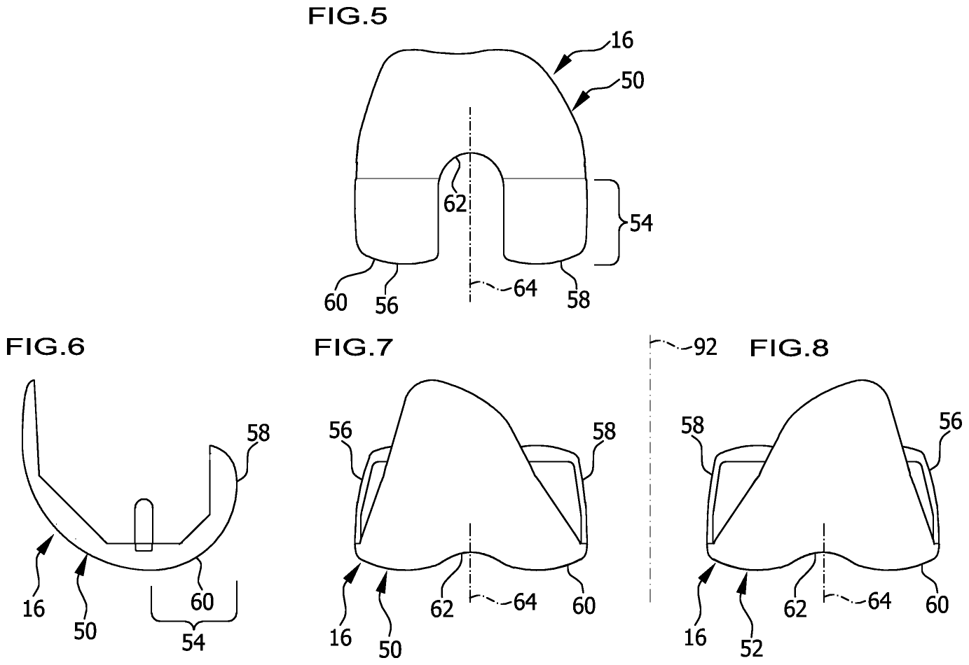

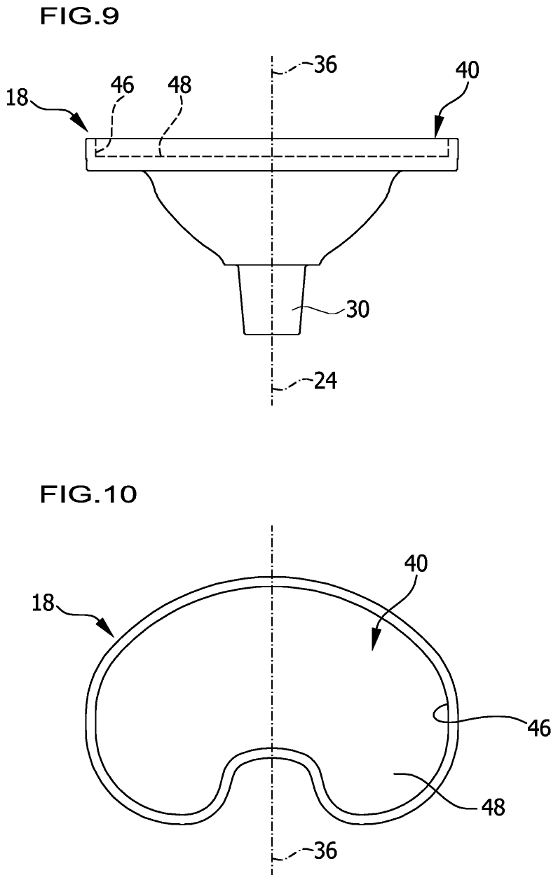

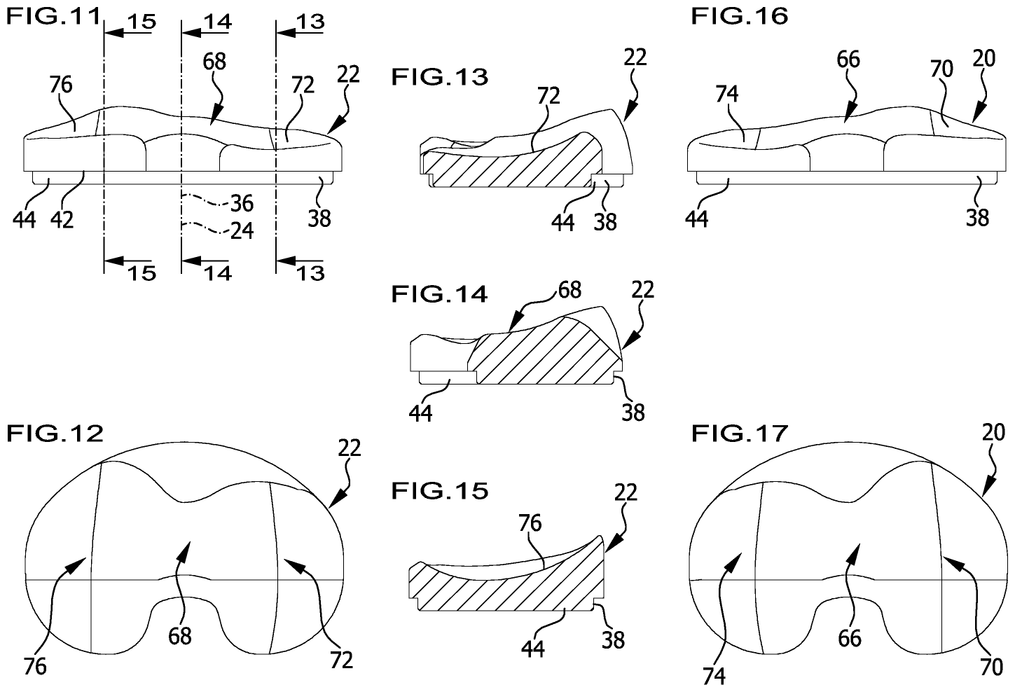

At this point it should be noted that in FIGS. 1 to 4 the tibia 28 and the associated tibial component 18 are depicted rotated by 180° relative to the femur 32 and the femoral component 16 and the femoral components 50 and 52 with respect to the prosthesis longitudinal direction. In these Figures the tibia 28 and the tibial component 18 are thus depicted from behind, the femur 32 and the femoral component 16 and the femoral components 50 and 52 from the front. A better view of the meniscal component sliding faces 64 and 66 of the meniscal components 20 and 22 is thus made possible. The crosswise association of the sliding face regions 70 and 72 with the condyles 56 and 58, as described above in connection with FIG. 3, is also the case for the schematic depictions in FIGS. 1, 2, and 4.

In the embodiment of the knee joint endoprosthesis set 10 depicted in FIG. 1, the medial sliding face region 70 and the lateral sliding face region 74 of the meniscal component 20 differ from one another in shape and size. Furthermore, the shape and size of the medial sliding face region 72 and of the lateral sliding face region 76 differ from one another.

The medial condyle 56 and the medial sliding face region 70 of the meniscal component 20 define a medial joint region 86. The lateral condyle 58 and the lateral sliding face region 74 define a lateral joint region 88.

One of the two joint regions 86 and 88 has a higher congruency between the condyle 56 or 58 and the associated sliding face region 70 or 74 than the other joint region.

In the embodiment depicted in FIG. 1, the congruency between the medial sliding face region 70 and the medial condyle 56 is greater than the congruency between the lateral condyle 58 and the lateral sliding face region 74. If for forming the knee joint endoprosthesis 14 the meniscal component 20 is configured in combination with the femoral component 16 in the form of the left femoral component 50 and with the tibial component 18, a movement pattern according to the “medial pivot” design can thus be achieved. The meniscal component 20 is hereby immovably held on the tibial component 18. In particular, it cannot be moved relative to the tibial component 18 in a plane that extends perpendicularly to the prosthesis longitudinal direction 24. The knee joint endoprosthesis 14 is thus of the “fixed bearing” type.

When the left femoral component 50 is combined with the meniscal component 22, the congruency between the lateral condyle 58 and the lateral sliding face region 76 is greater than between the medial condyle 56 and the medial sliding face region 72. A knee joint endoprosthesis 14 with the left femoral component 50, the meniscal component 22, and the tibial component 18 is therefore suited to define a movement pattern according to the “lateral pivot” design. Here, too, the meniscal component 22 is immovably held on the tibial component 18. In particular, it cannot be moved relative to the tibial component 18 in a plane that extends perpendicularly to the prosthesis longitudinal direction 24. The knee joint endoprosthesis 14 is thus of the “fixed bearing” type in this case too.

In the embodiments described, the joint regions 86 and 88 with the higher congruency define a ball jointed or substantially ball jointed joint region.

Schematically depicted in FIG. 2 is a knee joint endoprosthesis set 10 in which the femoral component 16 is configured in the form of a right femoral component 52. The two meniscal components 20 and 22 are identical to the meniscal components 20 and 22 of the embodiment of the knee joint endoprosthesis set 10 depicted in FIG. 1 with the left femoral component 50. When the right femoral component 52 is combined with the meniscal component 22, it results in a movement pattern of the “medial pivot” type for the knee joint endoprosthesis 14. If, however, the meniscal component 20 is used, the knee joint endoprosthesis 14 for replacing a right knee joint of a patient can achieve a movement pattern of the “lateral pivot” type. In both cases, the meniscal components 20 and 22 are immovably held on the tibial component 18. The knee joint endoprosthesis 14 formed is of the “fixed bearing” type.

In the described embodiments, the meniscal components 20 and 22 are configured in such a way that the first coupling portion 38 is of mirror symmetrical configuration relative to the coupling portion mirror plane 36, and the two sliding face regions 70 and 74 on the one hand and the two sliding face regions 72 and 76 on the other hand are of asymmetrical configuration relative to the coupling portion mirror plane 36.

Thus solely the cooperation of the meniscal components 20 and 22 having a respective meniscal component sliding face 66 and 68, which are asymmetrical as described, with the femoral component 16, the condyle portion 54 of which is configured mirror symmetrically to the femoral component mirror plane 64, predetermines the movement pattern of the knee joint endoprosthesis 14. It is thereby possible for a surgeon to still change the movement pattern for the knee at short notice during the surgical procedure, namely by swapping the meniscal components 20 and 22.

The predefinition of different movement patterns is possible both for knee joint endoprostheses 14 for the replacement of a left knee joint and for the replacement of a right knee joint. This is due to the fact that the condyle portions 54 both of the left femoral component 50 and of the right femoral component 52 are of identical configuration, in particular each being mirror symmetrical in themselves relative to the femoral component mirror plane 64.

The embodiments of knee joint endoprosthesis sets 10 depicted in FIGS. 1 and 2 form parts of a patient knee joint endoprosthesis set 90 for replacing any knee joint of the two knee joints of a patient.

The patient knee joint endoprosthesis set 90 comprises, e.g., the knee joint endoprosthesis set 10 depicted in FIG. 1 and also the right femoral component 52. Therefore, one of the two femoral components 16 of the patient knee joint endoprosthesis set 90 is configured in the form of a left femoral component 50 and the other is configured in the form of a right femoral component 52.

As already described in detail in connection with FIGS. 1 and 2, the two femoral components 16, i.e., the left femoral component 50 and the right femoral component 52, are combinable with the two meniscal components 20 and 22 as desired. Thus both right knee joints and left knee joints can be formed, namely, for example, both of the “medial pivot” type and of the “lateral pivot” type.

The patient knee joint endoprosthesis set 90 comprises, as described, a total of five components, namely two femoral components 16, namely a left femoral component 50 and a right femoral component 52, one single tibial component 18, and the two meniscal components 20 and 22.

The two femoral components 16 are configured mirror symmetrically to one another relative to a second mirror plane 92 extending in the prosthesis longitudinal direction 24. Said mirror plane 92 is indicated between the two femoral components 50 and 52 depicted in FIGS. 7 and 8. In other words, the left femoral component 50 can be converted into the right femoral component 92 by reflection on the second mirror plane 92.

In the embodiments depicted in the Figures, the first mirror plane 26 defines the second mirror plane 92. In other words, this means that a knee joint endoprosthesis 14 that is formed by the left femoral component 50, the meniscal component 20, and the tibial component 18 can be converted into a second knee joint endoprosthesis 14 by reflection on the first mirror plane 26 or the second mirror plane 92, said second knee joint endoprosthesis 14 being formed by the right femoral component 52, the meniscal component 22, and the tibial component 18.

In a further embodiment, the patient knee joint endoprosthesis set 90 comprises a further tibial component 18. The tibial component 18 may be used both for a left tibia and for a right tibia of the patient. Both tibial components 18 are therefore of identical configuration.

The knee joint endoprosthesis system 12 comprises, as described, at least one knee joint endoprosthesis set 10. Alternatively, the knee joint endoprosthesis system 12 comprises a patient knee joint endoprosthesis set 90.

The patient knee joint endoprosthesis set 90 enables a surgeon to replace one of the two knee joints of a patient with a knee joint endoprosthesis 14. The surgeon has all components required to replace both a right knee joint and a left knee joint and also the option of predetermining a movement pattern of the artificial knee as desired, namely by selecting one of the two meniscal components 20 or 22.

In a further embodiment, the knee joint endoprosthesis system 12 comprises two or more knee joint endoprosthesis sets 10. The femoral components 16 and the meniscal components 20 and 22 as well as the tibial components 18 of the two or more knee joint endoprosthesis sets 10 hereby differ in shape and/or size. This enables a surgeon to select from the knee joint endoprosthesis system 12 the femoral components and tibial components that are optimally suited for a patient with respect to their size as well as the suitable meniscal component, taking into account the desired movement pattern.

The described embodiments, in particular, of the knee joint endoprosthesis sets 10 enable a surgeon to still react flexibly to requirements during a surgical procedure for implanting the knee joint endoprosthesis 14, in particular with regard to the shape and size of the femoral component 16 and of the tibial component 18. In addition, it is possible for them to predetermine the desired movement pattern of the knee joint endoprosthesis 14 at short notice by appropriately selecting one of the two meniscal components 20 and 22.

1. A knee joint endoprosthesis set for replacing a knee joint of a patient, the knee joint endoprosthesis set comprising:
a knee joint endoprosthesis comprising:
a femoral component;
a tibial component; and
a first meniscal component;
a second meniscal component;
a coupling device for coupling the tibial component and the two meniscal components in a coupling position
wherein the first meniscal component and the second meniscal component are configured differently, wherein for forming the knee joint endoprosthesis, one of the first meniscal component and the second meniscal component is arranged between the femoral component and the tibial component cooperating therewith,
wherein the first meniscal component and the second meniscal component are configured mirror symmetrically to one another relative to a first mirror plane extending in a prosthesis longitudinal direction,
wherein the coupling device is of mirror symmetrical configuration relative to a coupling device mirror plane extending in a prosthesis longitudinal direction,
wherein the femoral component comprises a condyle portion with a medial condyle and a lateral condyle,
wherein each of the first meniscal component and the second meniscal component comprises a meniscal component sliding face cooperating with the medial condyle and the lateral condyle with a medial sliding face region and a lateral sliding face region,
wherein the medial condyle and the medial sliding face region form a medial sliding pairing,
wherein the lateral condyle and the lateral sliding face region form a lateral sliding pairing,
wherein at least one of a shape and a size of the medial sliding face region and of the lateral sliding face region differ from one another, and
wherein the condyle portion of the femoral component is of mirror symmetrical configuration relative to a femoral component mirror plane extending in a prosthesis longitudinal direction.

2. The knee joint endoprosthesis set according to claim 1, wherein the femoral component consists of only one single femoral component and the tibial component consists of only one single tibial component.

3. The knee joint endoprosthesis set according to claim 1, wherein the knee joint endoprosthesis set comprises only two meniscal components.

4. The knee joint endoprosthesis set according to claim 1, wherein the tibial component and one of the first meniscal component and the second meniscal component are coupled to one another in the coupling position for forming the knee joint endoprosthesis in such a way that said one of the first meniscal component and the second meniscal component is immovably held on the tibial component.

5. The knee joint endoprosthesis set according to claim 1, wherein the coupling device comprises two first coupling portions and a second coupling portion, wherein one of the two first coupling portions is arranged or formed on each of the first meniscal component and the second meniscal component, wherein the second coupling portion is arranged or formed on the tibial component, and wherein in the coupling position one of the two first coupling portions and the second coupling portion are at least one of in force-locking and positive-locking engagement with one another and in a separating position are out of engagement.

6. The knee joint endoprosthesis set according to claim 5, wherein the first coupling portion is of mirror symmetrical configuration relative to the coupling device mirror plane.

7. The knee joint endoprosthesis set according to claim 5, wherein the second coupling portion is of mirror symmetrical configuration relative to the coupling device mirror plane.

8. The knee joint endoprosthesis set according to claim 1, wherein the coupling device mirror plane defines the femoral component mirror plane.

9. The knee joint endoprosthesis set according to claim 1, wherein the medial condyle and the medial sliding face region define a medial joint region, wherein the lateral condyle and the lateral sliding face region define a lateral joint region, and wherein one of the medial joint region and the lateral joint region has a higher congruency between the condyle and the associated sliding face region than the other of the medial joint region and the lateral joint region.

10. The knee joint endoprosthesis set according to claim 9, wherein said one of the medial joint region and the lateral joint region defines a ball-jointed or substantially ball-jointed joint region.

11. The knee joint endoprosthesis set according to claim 1, wherein the first meniscal component comprises a first coupling portion for coupling to the tibial component and a meniscal component sliding face cooperating with the femoral component with a medial sliding face region and a lateral sliding face region, wherein the first coupling portion is of mirror symmetrical configuration relative to a coupling portion mirror plane, and wherein the two sliding face regions are of asymmetrical configuration relative to the coupling portion mirror plane.

12. The knee joint endoprosthesis set according to claim 11, wherein at least one of a shape and a size of the medial sliding face region and of the lateral sliding face region differ from one another.

13. A patient knee joint endoprosthesis set for replacing any knee joint of two knee joints of a patient, the patient knee joint endoprosthesis set comprising:
a knee joint endoprosthesis set for replacing a knee joint of a patient, comprising a knee joint endoprosthesis with a first femoral component, a first tibial component, and a first meniscal component,
wherein the knee joint endoprosthesis set comprises a second meniscal component, wherein the first meniscal component and the second meniscal component are configured differently,
wherein for forming the knee joint endoprosthesis, one of the first meniscal component and the second meniscal component is arranged between the first femoral component and the first tibial component cooperating therewith,
wherein the first meniscal component and the second meniscal component are configured mirror symmetrically to one another relative to a first mirror plane extending in a prosthesis longitudinal direction,
wherein the knee joint endoprosthesis set comprises a coupling device for coupling the first tibial component and the first meniscal component and the second meniscal component in a coupling position, wherein the coupling device is of mirror symmetrical configuration relative to a coupling device mirror plane extending in a prosthesis longitudinal direction,
wherein the first femoral component comprises a condyle portion with a medial condyle and a lateral condyle, wherein each of the first meniscal component and the second meniscal component comprises a meniscal component sliding face cooperating with the medial condyle and the lateral condyle with a medial sliding face region and a lateral sliding face region, wherein the medial condyle and the medial sliding face region form a medial sliding pairing, wherein the lateral condyle and the lateral sliding face region form a lateral sliding pairing,
wherein at least one of a shape and a size of the medial sliding face region and of the lateral sliding face region differ from one another, and
wherein the condyle portion of the first femoral component is of mirror symmetrical configuration relative to a femoral component mirror plane extending in a prosthesis longitudinal direction,
the patient knee joint endoprosthesis set further comprising:
a second femoral component, wherein the one of the first femoral component and the second femoral component is configured as a left femoral component and wherein the other of the first femoral component and the second femoral component is configured in the form of a right femoral component.

14. The patient knee joint endoprosthesis set according to claim 13, wherein the first femoral component and the second femoral component and the first meniscal component and the second meniscal component are configured to be combinable with one another as desired.

15. The patient knee joint endoprosthesis set according to claim 13, the first femoral component and the second femoral component are configured mirror symmetrically to one another relative to a second mirror plane extending in a prosthesis longitudinal direction.

16. The patient knee joint endoprosthesis set according to claim 15, wherein the first mirror plane defines the second mirror plane.

17. The patient knee joint endoprosthesis set according to claim 13, wherein the patient knee joint endoprosthesis set comprises a second tibial component.

18. The patient knee joint endoprosthesis set according to claim 17, wherein the first tibial component and the second tibial component are of identical configuration.

19. A knee joint endoprosthesis system comprising:
a) at least one knee joint endoprosthesis set for replacing a knee joint of a patient, comprising a knee joint endoprosthesis with a femoral component, a tibial component, and a first meniscal component, wherein the at least one knee joint endoprosthesis set comprises a second meniscal component, wherein the first meniscal component and the second meniscal component are configured differently, wherein for forming the knee joint endoprosthesis, one of the first meniscal component and the second meniscal component is arranged between the femoral component and the tibial component cooperating therewith,
wherein the first meniscal component and the second meniscal component are configured mirror symmetrically to one another relative to a first mirror plane extending in a prosthesis longitudinal direction,
wherein the at least one knee joint endoprosthesis set comprises a coupling device for coupling the tibial component and the first meniscal component and the second meniscal component in a coupling position, wherein the coupling device is of mirror symmetrical configuration relative to a coupling device mirror plane extending in a prosthesis longitudinal direction,
wherein the femoral component comprises a condyle portion with a medial condyle and a lateral condyle, wherein each of the first meniscal component and the second meniscal component comprises a meniscal component sliding face cooperating with the medial condyle and the lateral condyle with a medial sliding face region and a lateral sliding face region, wherein the medial condyle and the medial sliding face region form a medial sliding pairing, wherein the lateral condyle and the lateral sliding face region form a lateral sliding pairing,
wherein at least one of a shape and a size of the medial sliding face region and of the lateral sliding face region differ from one another, and
wherein the condyle portion of the femoral component is of mirror symmetrical configuration relative to a femoral component mirror plane extending in a prosthesis longitudinal direction,
or
b) a patient knee joint endoprosthesis set for replacing any knee joint of two knee joints of a patient, comprising:
a knee joint endoprosthesis set for replacing a knee joint of a patient, the knee joint endoprosthesis set comprising a knee joint endoprosthesis with a first femoral component, a tibial component, and a first meniscal component, wherein the knee joint endoprosthesis set comprises a second meniscal component, wherein the first meniscal component and the second meniscal component are configured differently, wherein for forming the knee joint endoprosthesis, one of the first meniscal component and the second meniscal component is arranged between the first femoral component and the tibial component cooperating therewith,
wherein the first meniscal component and the second meniscal component are configured mirror symmetrically to one another relative to a first mirror plane extending in a prosthesis longitudinal direction,
wherein the knee joint endoprosthesis set comprises a coupling device for coupling the tibial component and the first meniscal component and the second meniscal component in a coupling position, wherein the coupling device is of mirror symmetrical configuration relative to a coupling device mirror plane extending in a prosthesis longitudinal direction,
wherein the first femoral component comprises a condyle portion with a medial condyle and a lateral condyle, wherein each of the first meniscal component and the second meniscal component comprises a meniscal component sliding face cooperating with the medial condyle and the lateral condyle with a medial sliding face region and a lateral sliding face region, wherein the medial condyle and the medial sliding face region form a medial sliding pairing, wherein the lateral condyle and the lateral sliding face region form a lateral sliding pairing,
wherein at least one of a shape and a size of the medial sliding face region and of the lateral sliding face region differ from one another,
wherein the condyle portion of the first femoral component is of mirror symmetrical configuration relative to a femoral component mirror plane extending in a prosthesis longitudinal direction, and
wherein the patient knee joint endoprosthesis set further comprises a second femoral component, wherein the one of the first femoral component and the second femoral component is configured as a left femoral component and wherein the other of the first femoral component and the second femoral component is configured as a right femoral component.

20. The knee joint endoprosthesis system according to claim 19, further comprising at least two knee joint endoprosthesis sets and wherein at least one of the femoral component, the first meniscal component, and the tibial component of the at least two knee joint endoprosthesis sets differ at least one of in shape and size.
